(19) United States
(12) Patent Application Publication
(10) Pub. No.: US 2011/0186057 A1
(43) Pub. Date: Aug. 4, 2011

(54) ICU/CCU/NICU PATIENT GOWN

(76) Inventor: Richard Hatton, Okemos, MI (US)

(21) Appl. No.: 12/700,403

(22) Filed: Feb. 4, 2010

(51) Int. Cl. A61F 13/00; A41D 13/12

(52) U.S. Cl. 128/889; 2/83; 2/114

(57) ABSTRACT

An ICU/CCU/NICU garment for a patient during hospitalization includes an upper portion of the garment, a lower portion of the garment, and a rear portion of the garment. The upper garment portion includes a pair of front panels releasably connected to each other and to a neck panel leaving an open back. The lower portion of the garment includes an abdominal panel having a pair of leg extensions and a central genital panel extending downwardly therefrom leaving an open back. The rear portion having a rear panel is releasably attached to each of the upper portion and the lower portion to provide a desired degree of coverage, while facilitating a selective exposure of the posterior of the patient.

### FIELD OF THE INVENTION

The invention relates to a garment worn by a patient during a stay in an intensive care unit (ICU), coronary care unit (CCU), or a neurointensive care unit (NICU) of a hospital.

### BACKGROUND OF THE INVENTION

The requirements for hospital gowns in ICU/CCU/NICU settings are distinctly different than the requirements for conventional hospital gowns. Known hospital gowns substantially encircle the patient. When a caregiver such as a physician, a nurse, or ancillary personnel (e.g., a laboratory technician, a radiology technician, or a physical therapist) conducts a care procedure on a patient in an ICU/CCU/NICU setting, convenient access to the patient's body is often essential. However, there needs to be a balance between access to the patient's body, and the patient's desire for modesty and dignity when the patient is being dangled at the bedside or minimally ambulated, for example, prior to a transfer to a medical/surgical unit.

Furthermore, ICU/CCU/NICU gowns that encircle the patient have a potential to gather or “bunch up” between the patient and a bed surface. The bunching up of the gown material undesirably results in pressure points on the posterior of the patient. Skin irritation and the potential for skin breakdown and bed sores can result from pressure points when the patient is bedridden over a period of time, for example, when the patient is severely ill or comatose. With natural exposure to microorganisms in the hospital environment, and the subsequent infections that result therefrom, serious conditions can arise that may further compromise the patient's health.

There is a continuing need for an ICU/CCU/NICU gown for a patient who is essentially bed bound, but may be minimally ambulated. Desirably, the ICU/CCU/NICU gown provides a balance between caregiver access to the patient and the patient's modesty concerns, and minimizes the potential for infection due to skin irritation and breakdown.

### SUMMARY OF THE INVENTION

In concordance with the instant disclosure, an ICU/CCU/NICU gown for a patient who is essentially bed bound, but may ambulated minimally, for example, prior to a transfer to a medical/surgical unit, and which provides a balance between caregiver access to the patient and the patient's modesty concerns, and minimizes the potential for infection due to skin irritation and breakdown, is surprisingly discovered.

The invention includes a garment worn by a patient during a hospital stay in an ICU/CCU/NICU setting. The garment provides accessibility to various parts of the patient's body while providing a satisfying amount of coverage for the patient.

The garment is composed of a single layer of material. The single layer of material is cut in such a manner so that an upper portion is divided into two panels covering the upper torso of the patient. The upper portion includes a left upper panel and a right upper panel. The left upper panel has a neck panel that is permanently affixed thereto. The neck panel goes behind the patient's neck and is releasably attached to the right upper panel by a snap.

The left and right upper panels of the upper portion of the garment are cut at angles from the shoulders and meet at approximately the mid-breast bone area of the patient. The left and right upper panels of the upper portion are releasably attached by a snap at the mid-breast bone area of the patient. The left and right upper panels cooperate with the neck panel to form an opening for the head and neck of the patient.

The garment also includes a lower portion having an abdominal panel at a midline, e.g., a mid-hip level or a navel level, of the patient. The left and right upper panels of the upper portion are releasably attached by snaps at their respective inferior edges to a superior edge of the abdominal panel. The abdominal panel also has two leg extensions extending downwardly therefrom to cover the fronts of the legs of the patient. The leg extensions terminate at hemlines disposed approximately at a mid-calf level of the patient's legs. Pairs of releasable snaps are disposed at the mid-calf level of each of the leg extensions. The pairs of releasable snaps can be mated to cause each of the leg panels to encircle the hemline only of each mid-calf region over which they are respectively disposed.

The garment also includes a rear portion having a rear panel formed from a single sheet of material. The rear panel releasably attaches to the left and right upper panels of the upper portion. In addition, releasable snaps are located on each side of the abdominal panel at approximately the hip level, and can be mated to snaps at approximately the hip level on each side of the rear panel. The rear panel of the gown terminates at approximately the mid-calf of the patient. The rear panel is used only when the patient is dangled at bedside or ambulated in order to provide adequate coverage of the back of the patient. When the patient is lying in bed, the rear panel may be folded and laid on the bed beside the patient, or hung over a side of the bed.

In one embodiment, a garment to be worn by a patient during hospitalization in an ICU/CCU/NICU setting includes an upper portion adapted to cover an anterior upper body of the patient. The upper portion includes a pair of upper panels, each of the upper panels having a superior edge, an inferior edge, a side edge, and a central edge. Each of the central edges has an angled portion extending downwardly from the superior edge and away from the side edge to a point approximately midway between the superior edge and the inferior edge of each of the upper panels. One of the upper panels has an elongate rear neck panel permanently affixed to the superior edge thereof and extending from the superior edge of the one of the upper panels toward the superior edge of the other upper panel. The neck panel has a free end releasably attached to the superior edge of the other of the upper panels with a cooperating fastener. The neck panel and the front panels define an opening for receiving a head and a neck of the patient when the front panels are releasably attached at the central edges of the front panels with a cooperating fastener, and the end of the neck panel is releasably attached to the superior edge of the other of the upper panels with the first cooperating fastener.

The garment also includes a lower portion adapted to cover an anterior lower body of the patient. The lower portion has an abdominal panel with a superior edge at a midline level or approximately the level of the navel of the patient. Extending from the inferior edge are two leg panels. The leg panels have hemlines terminating at approximately the mid-calf level of the patient's legs. Each of the central edges extends upwardly from one of the inferior edges and away from the side edges to an intersection point between a genital area of the patient and the superior edge of the abdominal panel. The central edges, the side edges, and the inferior edges of the abdominal panel define a pair of leg extensions configured to cover the front legs of the patient. The side edges of the abdominal panel, in combination with central edges, the side edges, and the hemline edges of the leg panels are configured to cover the lower abdominal region and leg areas to the mid-calf levels of the patient. The superior edge of the abdominal panel is releasably attached to the inferior edges of each of the upper panels at the midline with a pair of releasable fasteners. There are cooperating releasable fasteners at the inferior edges of each leg panel at opposite corners. These fasteners will enclose the hemline only region of the leg panels and are designed to maintain leg coverage in the event of spontaneous leg motion.

The lower portion of the garment further includes a genital panel fixedly attached at the superior edge of the abdominal panel and extending downwardly to an area between the intersection point of the leg extensions and the inferior edges of the leg extensions. A remainder of the genital panel is free to move with respect to the abdominal panel and the leg extensions, and is arranged so as to overlap the intersection point between the genital area of the patient and the superior edge of the abdominal panel. The genital panel thereby provides a modest coverage for the patient.

The garment also includes a rear portion adapted to selectively cover a posterior of the patient. The rear portion includes a rear panel having an upper edge, a lower edge, and a pair of side edges. The side edges of the rear panel are releasably attached to the side edges of one of the upper portion and the lower portion at the mid-hip level of the patient with cooperating fasteners. Opposite corners of the upper edge of the rear panel are also releasably attached to the superior edges of the upper panels of the upper portion with cooperating fasteners.

The garment completely exposes a posterior of the patient from a shoulder level of the patient to a mid-calf level of the patient when worn by the patient with the rear panel open. The garment also completely covers the posterior of the patient from the shoulder level of the patient to the mid-calf level of the patient when worn by the patient with the rear panel closed.

In another embodiment, a method for militating against skin irritation and sores in a patient during a hospital stay in an ICU/CCU/NICU setting includes the steps of: providing the garment as described herein; covering the posterior of the patient by closing the rear panel when the patient is dangled at a bed or ambulated; exposing the posterior of the patient from the shoulder level of the patient to the mid-calf level of the patient by opening the rear panel when the patient is lying in the bed; and one of folding the rear panel and laying the rear panel on the bed beside the patient, and disposing the rear panel over a side of the bed beside the patient while the patient is lying in the bed.

### DRAWINGS

The above, as well as other advantages of the present invention, will become readily apparent to those skilled in the art from the following detailed description of the invention when considered in the light of the accompanying drawings in which:

FIG. 1 is a front view of a garment according to the present disclosure, illustrating an upper portion, a lower portion, and a rear portion;

FIG. 2 is a front view of the garment illustrated in FIG. 1, showing the garment worn by a patient with the rear panel closed;

FIG. 3 is a rear view of the garment illustrated in FIGS. 1 and 2, showing the garment worn by the patient with the rear panel closed;

FIG. 4 is a rear view of the garment illustrated in FIGS. 1-3, showing the garment worn by the patient with the rear panel opened to completely expose the posterior of the patient from the shoulder level to the mid-calf level of the patient;

FIG. 5 is an enlarged, partial rear view of the garment illustrated in FIGS. 1-4, showing the snapping of a leg panel to encircle the mid-calf region of the patient's legs at the hemline only; and

FIG. 6 is a top perspective view of the garment illustrated in FIGS. 1-5, showing the patient lying in a bed with the rear portion of the garment opened and hung over a side of the bed.

### DETAILED DESCRIPTION OF THE INVENTION

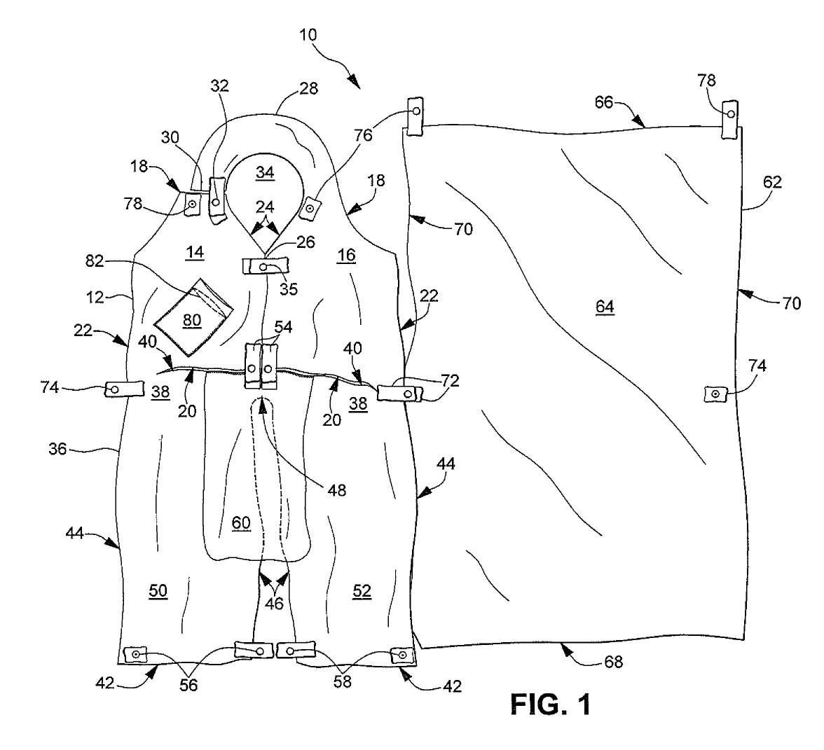

Referring now to FIGS. 1-6, a garment 10 for a patient 11 in a hospital is shown. The garment 10 is advantageously designed to be worn by or placed upon a body of the patient 11 when the patient 11 is hospitalized in a coronary care unit (CCU), an intensive care unit (ICU), a neurointensive care unit (NICU), or the like.

The garment 10 includes an upper portion 12 adapted to cover an anterior upper body of the patient 11. The upper portion 12 has a pair of upper panels 14, 16, including a right upper panel 14 and a left upper panel 16. Each of the upper panels 14, 16 has a superior edge 18, an inferior edge 20, a side edge 22, and a central edge 24. The central edges 24 have angled portions that extend downwardly form the respective superior edges 18 of the upper panels 14, 16, and away form the respective side edges 22 of the upper panels 14, 16. The central edges 24 extend to a point 26 approximately midway between the superior edges 18 and the inferior edges 20 of the upper panels 14, 16. For example, the angled portions of the central edges 24 may extend to about a mid-breast bone level of the patient 11.

The upper portion 12 of the garment 10 also includes an elongate neck panel 28 adapted to cover a rear of a neck of the patient 11. The neck panel 28 is permanently affixed to the superior edge 18 of one of the upper panels 14, 16, for example, the left upper panel 16 shown in FIG. 1. The neck panel 28 may be integrally formed with the one of the upper panels 14, 16, or permanently affixed by stitching, for example. Other means for permanently affixing the neck panel 28 to the one of the upper panels 14, 16 may also be employed. The neck panel 28 extends from the superior edge 18 of the one of the upper panels 14, 16 toward the other of the upper panels 14, 16. A free end 30 of the neck panel 28 is releasably secured to the superior edge 18 of the other of the upper panels 14, 16 with first cooperating fasteners 32.

The neck panel 28 and the pair of front panels 14, 16 define an opening 34 of the upper portion 12 when the front panels 14, 16 are releasably attached at the central edges 24 with second cooperating fasteners 35, and the neck panel 28 is releasably attached to the other of the front panels 14, 16 with the first cooperating fasteners 32. The opening 34 is adapted to receive the head and the neck of the patient 11 when the garment 10 is worn by the patient 11. When donned by or placed on the patient 11, the upper portion 12 of the garment 10 provides a satisfying amount of coverage of the front or anterior of the upper body of the patient 11 while also allowing doctors and nurses access via the releasably attached front panels 14, 16 for treatment and care of the patient 11.

The garment 10 further includes a lower portion 36 adapted to cover an anterior lower body of the patient 11. The lower portion 36 may be formed separate from, or be integral with, the upper portion 12 of the garment 10, as desired. The lower portion 36 has an abdominal panel 38 with a superior edge 40 disposed at about a navel level of the patient 11. The abdominal panel 38 may extend generally horizontally at waist level across the front of the patient 11, for example, below an upper hip or navel level point of the patient 11. The abdominal panel 38 also has a pair of inferior edges 42 disposed at a mid-calf level of the patient 11, a pair of side edges 44, and a pair of central edges 46. Each of the central edges 46 extend upwardly from one of the inferior edges 42 and away from the side edges 44 to an intersection point 48. The intersection point 48 is between a genital area of the patient 11 and the superior edge 40 of the abdominal panel 38. As shown in FIG. 1, the intersection point 48 may be at an apex of a substantially inverse U-shaped edge portion connecting the pair of central edges 46.

The central edges 46, the side edges 44, and the inferior edges 42 of the abdominal panel 38 define a pair of leg extensions 50, 52, including a right leg extension 50 and a left leg extension 52. The leg extensions 50, 52 are adapted to cover the fronts of the legs of the patient 11 when the garment 10 is worn by the patient 11

The superior edge 40 of the abdominal panel 38 is releasably attached to the inferior edges 20 of each of the upper panels 14, 16 with a pair of third cooperating fasteners 54. Fourth cooperating fasteners 56, 58 are disposed at the inferior edges 42 of each of the leg extensions 50, 52. The inferior edge 42 of the right leg extension 50 has the fourth cooperating fasteners 56 spaced apart and disposed at opposite corners of the inferior edge 42. Likewise, the inferior edge 42 of the left leg extension 52 has the fourth cooperating fasteners 58 spaced apart and disposed at opposite corners of the inferior edge 42. The fourth cooperating fasteners 56, 58 may be releasably attached to permit each of the leg extensions 50, 52 to wrap around the leg at the hemline only of the patient 11 at the mid-calf level of the patient 11. It should be understood that the fourth cooperating fasteners 56, 58 are configured such that the rears of the legs of the patient 11 are completely exposed above the mid-calf level when the fourth cooperating fasteners 56, 58 are releasably attached to encircle the legs.

The lower portion 36 of the garment 10 also includes a genital panel 60. The genital panel 60 is fixedly attached at the superior edge 40 of the abdominal panel 38, for example, by sewing. The genital panel 60 extends downwardly from the superior edge 40 to an area between the intersection point 46 of the leg extensions 50, 52 and the inferior edges 42 of the leg extensions 50, 52. The remainder of the genital panel 60 is free to move with respect to the abdominal panel 38 and the leg extensions 50, 52. The genital panel 60 is arranged to overlap at least the intersection point 48 between the genital area of the patient 11 and the superior edge 40 of the abdominal panel 38 in order to provide modest coverage for the patient 11 when the garment 10 is worn by the patient 11. It should be appreciated that the genital panel 60 may be lifted to allow doctors and nurses access to provide treatment and care to the patient 11, as desired.

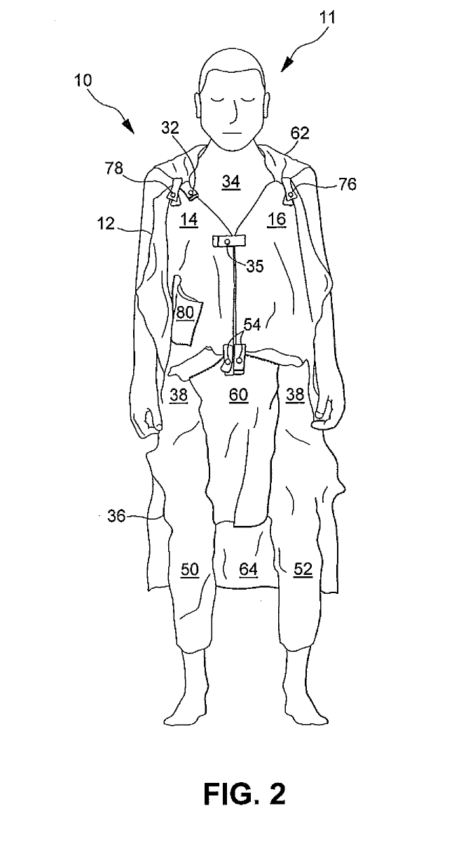

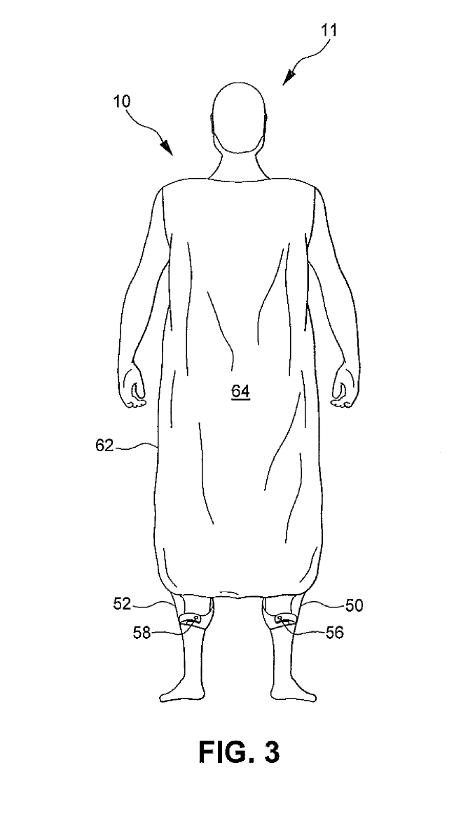

The garment 10 additionally includes a rear portion 62 adapted to selectively cover a posterior of the patient 11. The rear portion 62 includes a rear panel 64 having an upper edge 66, a lower edge 68, and a pair of side edges 70. As a nonlimiting example, the rear panel 64 may be substantially rectangular, although other suitable shapes may also be employed. The side edges 70 of the rear panel 64 are releasably attached to the side edges 22, 44 of one of the upper portion 12 and the lower portion 36 at approximately the mid-hip level of the patient 11 with fifth cooperating fasteners 72 and sixth cooperating fasteners 74. Seventh and eighth cooperating fasteners 76, 78 are also disposed at opposite corners of the upper edge 66 of the rear panel 64, and at the superior edges 18 of the right and left upper panels 14, 16. The opposite corners of the upper edge 66 of the rear panel 64 are thereby releasably attached to the superior edges 18 of the upper portion 12 at about the shoulder level of the patient 11.

Where it is desired that the posterior of the patient 11 be completely covered, for example, as shown in FIGS. 2 and 3, one of the side edges 70 of the rear panel 64 may be attached to the one of the side edges 44 of the lower portion 36 at approximately the mid-hip level of the patient 11. The other of the side edges 70 may also be attached to the other of the side edges 44 of the lower portion 36 at approximately the mid-hip level of the patient 11. Likewise, the opposite corners of the upper edge 66 of the rear panel 64 may be attached to the superior edges 18 of the upper portion 12 of the garment 10. The rear panel 64 is thereby closed and the posterior of the patient 11 is completely covered.

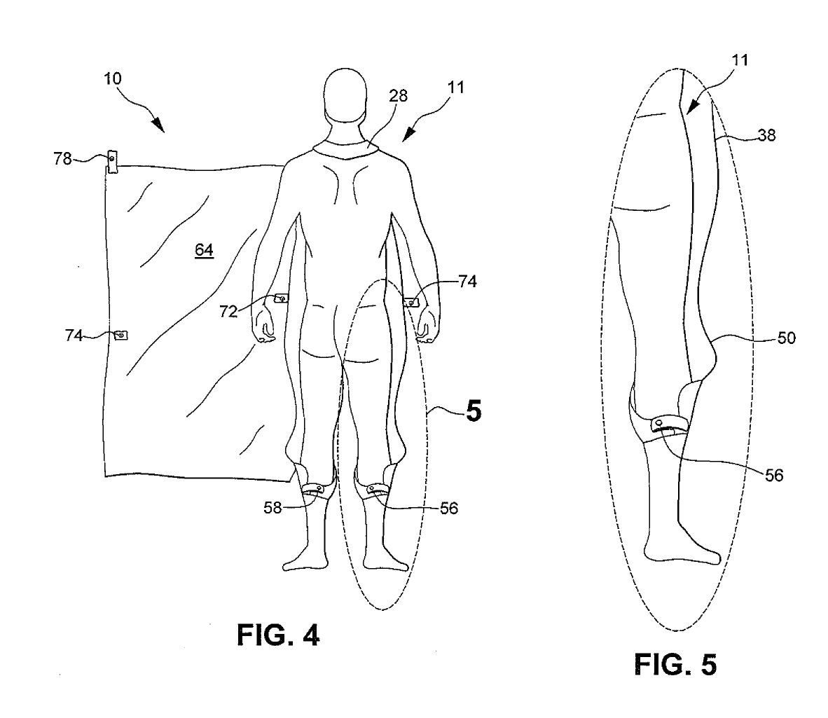

In a further embodiment, where it is desired that the posterior of the patient 11 be completely exposed from the shoulder level of the patient 11 to the mid-calf level of the patient 11, for example, as shown in FIGS. 4-6, the opposite corners of the upper edge 66 of the rear panel 64 may be released from the superior edges 18 of the upper portion of the garment. Additionally, the one of the side edges 70 of the rear panel 64 may be released from the one of the side edges 44 of the lower portion 36 at the mid-hip level of the patient 11, to thereby expose the posterior of the patient 11. The rear panel 64 is thereby opened and the posterior of the patient 11 is completely exposed.

A pouch panel 80 may be attached to one of the right and left front panels 14, 16 of the garment 10. The pouch panel 80 may be generally rectangular-shaped and is preferably attached to one of the right and left front panels 14, 16 at two side edges and a bottom edge thereof to define a pouch. The pouch is sized to receive and/or contain a telemetry pack and leads (not shown) for the patient 11. A slit 82 may further be formed in one of the right and left front panels 14, 16 behind the pouch panel 80 to permit the telemetry leads to pass to the patient 11.

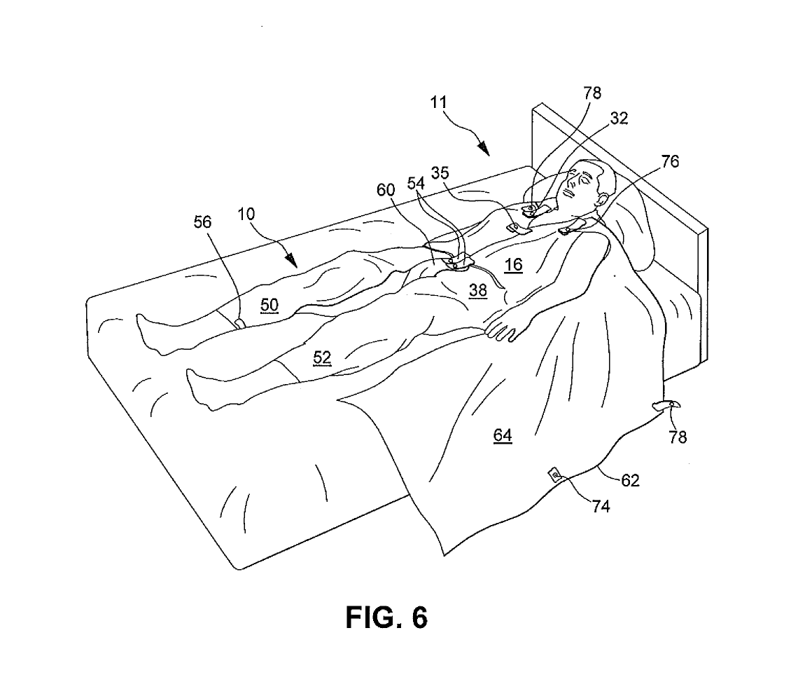

As shown in FIG. 6, when the garment 10 is placed on the patient 11 lying in the bed on his/her back, the garment 10 provides a satisfying amount of coverage for the front of the body of the patient 11, while advantageously allowing doctors and nurses convenient access to a variety of body parts, for the purpose of providing treatment and care to the patient 11.

As described hereinabove, the garment 10 has a plurality of the fasteners, e.g., the cooperating fasteners 32, 35, 54, 56, 58, 72, 74, 76, 78, configured to selectively hold the garment 10 on the patient 11, for example, when the patient 11 is either dangled or ambulated. The fasteners employed with the garment 10 of the present disclosure may each include a male fastener and a female fastener, for example. In a particular embodiment, each of the fasteners may be a male-female snap, with one of the male portion and the female portion attached to a strip having one end fixedly attached garment 10, and the other of the male portion and the female portion fixedly attached to the garment 10. The strip may thereby provide for a selective opening and closing of the garment 10. Other suitable fasteners may be, but are not limited to, buttons and button holes, other snap designs, cooperating hook and loop strips, and the like. The locations and number of fasteners shown in the figures and described above are illustrative only and are not limiting. One of ordinary skill in the art may select suitable fasteners and locations for the fasteners, as desired.

The garment 10 may be formed from any desired material, including a disposable material, a biodegradable material, or a machine-washable cloth conventionally used for hospital garments, as nonlimiting examples.

The present disclosure also includes a method for militating against skin irritation and sores in the patient 11 during a hospital stay in an ICU/CCU/NICU setting. The method includes the steps of: providing the garment 10 as described hereinabove; covering the posterior of the patient 11 by closing the rear panel 64 when the patient 11 is dangled at the bed or ambulated; exposing the posterior of the patient 11 from the shoulder level of the patient 11 to the mid-calf level of the patient 11 by opening the rear panel 64 when the patient is lying in the bed; and one of folding the rear panel 64 and laying the rear panel 64 on the bed beside the patient 11, and disposing the rear panel 64 over a side of the bed beside the patient 11 while the patient 11 is lying in the bed

It should be appreciated that the rear panel 64 of the garment 10 may be folded and laid on the bed beside the patient 11, or hung over a side of the bed as shown in FIG. 6, when the patient 11 is lying in the hospital bed. When configured thusly, the rear of the body of the patient 11 is completely exposed. A formation of pressure points due to folded fabric underneath the patient 11, and the occurrence of related skin irritations and bed sores, is thereby militated against. Where it is desired that the patient be dangled on the bed, or ambulated, the rear panel 64 may be disposed over the rear of the body of the patient 11, and thereby provide a desired amount of coverage for the patient 11.

Advantageously, when the garment 10 is placed on the patient 11 lying in the bed on his/her back, the garment 10 provides a satisfying amount of coverage for the front of the body of the patient 11, allows doctors and nurses access to provide treatment and care to the patient 11, and militates against the formation of bed sores and related infections in the patient 11.

In accordance with the provisions of the patent statutes, the present invention has been described in what is considered to represent its preferred embodiment. However, it should be noted that the invention can be practiced otherwise than as specifically illustrated and described without departing from its spirit or scope.

What is claimed is:

1. A garment to be worn by a patient during hospitalization in an ICU/CCU/NICU setting, the garment comprising:
an upper portion adapted to cover an anterior upper body of the patient, the upper portion including a pair of upper panels, each of the upper panels having a superior edge, an inferior edge, a side edge, and a central edge, each of the central edges having an angled portion extending downwardly from the superior edge and away from the side edge to a point approximately midway between the superior edge and the inferior edge of each of the upper panels, one of the upper panels having an elongate rear neck panel permanently affixed to the superior edge thereof and extending from the superior edge of the one of the upper panels toward the other of the other upper panels, the neck panel having a free end releasably attached to the superior edge of the other of the upper panels with a first cooperating fastener, the neck panel and the front panels defining an opening for receiving a head and a neck of the patient when the front panels are releasably attached at the central edges of the front panels with a second cooperating fastener and the end of the neck panel is releasably attached to the superior edge of the other of the upper panels with the first cooperating fastener;
a lower portion adapted to cover an anterior lower body of the patient, the lower portion having an abdominal panel with a superior edge disposed at approximately a navel level of the patient, a pair of inferior edges disposed at a mid-calf level of the patient, a pair of side edges, and a pair of central edges, each of the central edges extending upwardly from one of the inferior edges and away from the side edges to an intersection point between a genital area of the patient and the superior edge of the abdominal panel, the central edges, the side edges, and the inferior edges of the abdominal panel defining a pair of leg extensions configured to cover the front legs of the patient, the superior edge of the abdominal panel releasably attached to the inferior edges of each of the upper panels with a pair of third fasteners, the inferior edges of each of the leg extensions having fourth cooperating fasteners disposed at opposite corners thereof, the fourth cooperating fasteners being releasably attached to permit each of the leg extensions to encircle a leg of the patient at mid-calf level at only a hemline of the leg extension, the lower portion of the garment further including a genital panel fixedly attached at the superior edge of the abdominal panel and extending downwardly to an area between the intersection point of the leg extensions and the inferior edges of the leg extensions, the remainder of the genital panel being free to move with respect to the abdominal panel and the leg extensions and arranged so as to overlap the intersection point between the genital area of the patient and the superior edge of the abdominal panel and provide a modest coverage for the patient; and
a separate rear portion adapted to selectively cover a posterior of the patient, the rear portion having a rear panel with an upper edge, a lower edge, and a pair of side edges, the side edges releasably attached to the side edges of one of the upper portion and the lower portion at the mid-hip level of the patient with fifth and sixth cooperating fasteners, and opposite corners of the upper edge of the rear panel releasably attached to the superior edges of the upper panels of the upper portion with seventh and eighth cooperating fasteners,
wherein the garment completely exposes a posterior of the patient from a shoulder level of the patient to a mid-calf level of the patient when worn by the patient with the rear panel of the rear portion open, and completely covers the posterior of the patient from the shoulder level of the patient to the mid-calf level of the patient when worn by the patient with the rear panel of the rear portion closed.

2. The garment according to claim 1, wherein the second cooperating fastener for releasably attaching the central edges of the front panels includes a strip fixedly attached to one of the front panels, the strip having the fastener attached to an end thereof for releasable attachment to the other of the front panels.

3. The garment according to claim 1, wherein each of the third cooperating fasteners for releasably attaching the abdominal panel to the front panels includes a strip fixedly attached to one of the front panels, the strip having the fastener attached to an end thereof for releasable attachment to the abdominal panel.

4. The garment according to claim 1, wherein each of the fourth cooperating fasteners disposed at the opposite corners of the inferior edges of each of the leg extensions includes a strip fixedly attached to one of the corners, the strip having the fastener attached to an end thereof for releasable attachment to the other of the corners of the leg extension at the hemline only.

5. The garment according to claim 1, wherein the rear portion is formed from a substantially rectangular, unitary panel of material.

6. The garment according to claim 1, including a pouch panel fixedly attached to one of the front panels and sized to receive a telemetry pack and a plurality of telemetry leads therein, a slit formed though the one of the front panels beneath the pouch panel through which the telemetry leads are provided to the patient.

7. The garment according to claim 1, wherein each of the cooperating fasteners includes one of a male-female snap, a hook and loop fastener, and a button and button hole.

8. The garment according to claim 1, wherein each of the cooperating fasteners includes a male-female snap.

9. The garment according to claim 1, wherein the neck panel is integrally formed with the one of the upper panels.

10. The garment according to claim 1, wherein the intersection point of the central edges of the leg extensions is disposed at an apex of a substantially U-shaped edge portion connecting the central edges.

11. A method for militating against skin irritation and sores in a patient during a hospital stay in an ICU/CCU/NICU setting, the method comprising the steps of:
providing a garment including an upper portion adapted to cover an anterior upper body of the patient, the upper portion including a pair of upper panels, each of the upper panels having a superior edge, an inferior edge, a side edge, and a central edge, each of the central edges having an angled portion extending downwardly from the superior edge and away from the side edge to a point approximately midway between the superior edge and the inferior edge of each of the upper panels, one of the upper panels having an elongate rear neck panel permanently affixed to the superior edge thereof and extending from the superior edge of the one of the upper panels toward the other of the other upper panels, the neck panel having a free end releasably attached to the superior edge of the other of the upper panels with a first cooperating fastener, the neck panel and the front panels defining an opening for receiving a head and a neck of the patient when the front panels are releasably attached at the central edges of the front panels with a second cooperating fastener and the end of the neck panel is releasably attached to the superior edge of the other of the upper panels with the first cooperating fastener, a lower portion adapted to cover an anterior lower body of the patient, the lower portion having an abdominal panel with a superior edge disposed at approximately a navel level of the patient, a pair of inferior edges disposed at a mid-calf level of the patient, a pair of side edges, and a pair of central edges, each of the central edges extending upwardly from one of the inferior edges and away from the side edges to an intersection point between a genital area of the patient and the superior edge of the abdominal panel, the central edges, the side edges, and the inferior edges of the abdominal panel defining a pair of leg extensions configured to cover the front legs of the patient, the superior edge of the abdominal panel releasably attached to the inferior edges of each of the upper panels with a pair of third fasteners, the inferior edges of each of the leg extensions having fourth cooperating fasteners disposed at opposite corners thereof, the fourth cooperating fasteners being releasably attached to permit each of the leg extensions to encircle a leg of the patient at mid-calf level at only a hemline of the leg extension, the lower portion of the garment further including a genital panel fixedly attached at the superior edge of the abdominal panel and extending downwardly to an area between the intersection point of the leg extensions and the inferior edges of the leg extensions, the remainder of the genital panel being free to move with respect to the abdominal panel and the leg extensions and arranged so as to overlap the intersection point between the genital area of the patient and the superior edge of the abdominal panel and provide a modest coverage for the patient, and a separate rear portion adapted to selectively cover a posterior of the patient, the rear portion having a rear panel with an upper edge, a lower edge, and a pair of side edges, the side edges releasably attached to the side edges of one of the upper portion and the lower portion at the mid-hip level of the patient with fifth and sixth cooperating fasteners, and opposite corners of the upper edge of the rear panel releasably attached to the superior edges of the upper panels of the upper portion with seventh and eighth cooperating fasteners, wherein the garment completely exposes a posterior of the patient from a shoulder level of the patient to a mid-calf level of the patient when worn by the patient with the rear panel of the rear portion open, and completely covers the posterior of the patient from the shoulder level of the patient to the mid-calf level of the patient when worn by the patient with the rear panel of the rear portion closed;
covering a posterior of the patient by closing the rear panel when the patient is dangled at a bed or ambulated;
exposing a posterior of the patient from a shoulder level of the patient to a mid-calf level of the patient by opening the rear panel when the patient is lying in the bed; and
one of folding the rear panel and laying the rear panel on the bed beside the patient, and disposing the rear panel over a side of the bed beside the patient while the patient is lying in the bed.
